(19) United States
(12) Patent Application Publication
(10) Pub. No.: US 2012/0115939 A1
(43) Pub. Date: May 10, 2012

(54) METHODS FOR USING LIPOIC ACID

(75) Inventors: Kimberly Ann Vonnahme, Fargo, ND (US); Erika Lynn Berg, Fargo, ND (US)

(73) Assignee: NDSU Research Foundation, Fargo, ND (US)

(21) Appl. No.: 13/292,525

(22) Filed: Nov. 9, 2011

Related U.S. Application Data
(60) Provisional application No. 61/411,749, filed on Nov. 9, 2010.

(51) Int. Cl. A61K 31/385; A61P 15/08; G01N 33/48; A61P 5/34

(52) U.S. Cl. 514/440; 73/61.43

(57) ABSTRACT

The present provides methods for using lipoic acid. In one embodiment, the methods include administering lipoic acid to a female subject in need thereof, wherein at least 10 milligrams lipoic acid per kilogram of body weight is administered. In another embodiment, the methods include administering to a female subject in need thereof an effective amount of lipoic acid, wherein concentration of progesterone in the blood of the subject is increased compared to the subject prior to administration of the lipoic acid. The subject may be pregnant.

### CROSS-REFERENCE TO RELATED APPLICATIONS

This application claims the benefit of U.S. Provisional Application Ser. No. 61/411,749, filed Nov. 9, 2010, which is incorporated by reference herein.

### BACKGROUND

Progesterone plays a major role in pregnancy, one being to promote production of endometrial secretions that aid in early development of the embryo (Geisert et al., 1992, Reprod. Fertil. Dev., 4:301-305). Low concentrations of progesterone can lead to poor embryonic development (Nephew et al., 1994, J. Anim. Sci., 72:453-458) and may enable an increase in hormones that cause embryonic death (Inskeep, 2004, J. Anim. Sci., 82:E24-39); however, it has been shown that supplementation of progesterone alone is not effective in overcoming progesterone deficiency.

### SUMMARY OF THE INVENTION

Fertility is an issue for all domestic livestock species, endangered species, and humans. There is tremendous economic loss associated with reduced fertility in livestock species. This increases the cost of maintaining livestock and consequently affects food availability and price. Improved fertility in zoo animals will potentially increase the number of animals born and reduce the number of species considered to be endangered.

The present invention provides methods for using lipoic acid. In one embodiment, the methods include administering lipoic acid to a subject in need thereof, wherein at least 10 milligrams lipoic acid per kilogram of body weight is administered. The lipoic acid may be present in a composition. The subject may be a female vertebrate, and in one embodiment is a human or a cow. The subject may be pregnant, and may be part of a breeding program. The administration may be oral, and may be administered as a single dosage, or administered over a 24 hour period.

In one embodiment, the methods include administering to a female subject in need thereof an effective amount of lipoic acid, wherein the concentration of progesterone in the blood of the subject is increased compared to the subject prior to administration of the composition. The lipoic acid may be present in a composition. In one embodiment, the concentration of progesterone in the blood may be increased to at least 2 ng/ml. The subject may be a vertebrate, and in one embodiment is a human or a cow. The subject may be pregnant, and may be part of a breeding program. The administration may be oral, and may be administered as a single dosage, or administered over a 24 hour period.

The present invention also provides methods for optimizing the therapeutic efficiency of lipoic acid treatment. In one embodiment, the methods include determining the level of progesterone in the subject administered lipoic acid. The subject may be one that has already received lipoic acid, or the method may further include administering lipoic acid to the subject prior to the determining. The method may further include obtaining a biological sample, such as blood, from the subject. A level of progesterone that is less than a desired concentration indicates a need to increase the amount of lipoic acid subsequently administered to the subject. Optionally, a level of progesterone that is greater than a desired concentration indicates a need to decrease the amount of lipoic acid subsequently administered to the subject. In one embodiment, a desired level of progesterone is 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml, or 50 ng/ml.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Average intercept for progesterone clearance in control and lipoic acid supplemented ovariectomized ewes after CIDR removal on day 11.

FIG. 2. Average rate constant for progesterone clearance in control and lipoic acid supplemented ovariectomized ewes after CIDR removal on day 11.

### DETAILED DESCRIPTION OF ILLUSTRATIVE EMBODIMENTS

The present invention includes administering to a subject lipoic acid, a derivative thereof, or a salt thereof. The lipoic acid may be present in a composition. Lipoic acid has the molecular formula C₈H₁₄O₂S₂, and is also known as alpha-lipoic acid, thioctic acid, and the IUPAC name (R)-5-(1,2-dithiolan-3-yl)pentanoic acid. Lipoic acid exists as two enantiomers, R-(+)-lipoic acid (R-lipoic acid) and S-(−)-lipoic acid (S-lipoic acid), and as a racemic mixture (R/S lipoic acid). Lipoic acid administered as described herein may be R-(+)-lipoic acid or R/S lipoic acid. Lipoic acid is commercially available from, for instance, MTC Industries, Inc (Edgewood, N.Y.). Derivatives of lipoic acid include compounds that can be formed by derivatization of lipoic acid, and have the same activity as lipoic acid as described herein, e.g., the ability to increase the level of progesterone in the blood of a subject. Unless stated otherwise, the term lipoic acid refers to lipoic acid, derivatives thereof, and salts thereof.

Compositions of the present invention may include a carrier, such as a pharmaceutically acceptable carrier. As used herein “pharmaceutically acceptable carrier” includes saline, solvents (e.g., a solution containing ethanol), dispersion media, coatings, antibacterial and antifungal agents, isotonic and absorption delaying agents, and the like, compatible with pharmaceutical administration. Additional active compounds can also be incorporated into the compositions.

A composition may be prepared by methods well known in the art of pharmaceutics. In general, a composition can be formulated to be compatible with its intended route of administration. Examples of routes of administration include parenteral, (e.g., intravenous, intradermal, subcutaneous), oral, transdermal (e.g., topical), transmucosal, and rectal administration. Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions (where water soluble) or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline (PBS). A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (i.e., lipoic acid) in the required amount in an appropriate solvent with one or a combination of ingredients, such as those enumerated above, as required, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying and freeze-drying which yields a powder of the active ingredient plus any additional desired ingredient from a previously sterilized solution thereof.

Oral compositions may include an inert diluent or an edible carrier. For the purpose of oral therapeutic administration, the active compound may be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. In one embodiment, administration by an oral route increases the uptake of lipoic acid by the subject before the lipoic acid is exposed to microbes present in, for instance, the stomach of a ruminant or the intestine of a monogastric.

In one embodiment, an edible carrier may be a food. For instance, when a composition is to be administered to a farm animal, the composition may be fed to the animal. Lipoic acid may be added to an animal's feed as a concentrated feed additive that is mixed with the feed or used to top dress a feed. For instance, the lipoic acid may be added to feed as a powder form or as a liquid that may then be allowed to dry on the feed before administration to an animal. In one embodiment, a carrier may be water. For instance, lipoic acid may be added to an animal's water supply. With some animals, for instance non-ruminants, the presence of sulfur in lipoic acid may be unpleasant to the animal and reduce uptake of the feed. In such cases, lipoic acid may be combined with other components to mask the sulfur, such as syrups, etc.

For administration by inhalation, the active compounds may be delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the bather to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions can also be used as pharmaceutically acceptable carriers. These can be prepared according to methods known to those skilled in the art.

The formulations may be conveniently presented in unit dosage form and may be prepared by methods well known in the art of pharmacy. Methods of preparing a composition with a pharmaceutically acceptable carrier include the step of bringing the active compound into association with a carrier that constitutes one or more accessory ingredients. In general, the formulations are prepared by uniformly and intimately bringing the active compound into association with a liquid carrier, a finely divided solid carrier, or both, and then, if necessary, shaping the product into the desired formulations. In general, a composition can be formulated to be compatible with its intended route of administration. More specifically, the compositions of the present invention may be administered to any tissue of a subject, including, but not limited to, muscle (such as skeletal muscle or cardiac muscle), skin, epithelial (e.g., mouth, esophageal), lung tissue, intestinal tissue, and the like. A composition of the present invention may be administered to any internal cavity of a subject, including, but not limited to, lungs, mouth, nasal cavity, stomach, peritoneal cavity, intestine, veins, and the like.

A composition of the present invention may be administered in an amount sufficient to permit, enhance, facilitate, and/or are conducive the methods described herein. For instance, the amount of lipoic acid present in a composition of the present invention can vary, and may depend on the dosage to be delivered and the route of administration. For an injectable composition (e.g. subcutaneous, intramuscular, etc.) the lipoic acid may be present in the composition in an amount such that the total volume of the composition administered is 0.5 ml to 5.0 ml, typically 1.0 to 2.0 ml. The amount administered may vary depending on various factors including, but not limited to, the form of lipoic acid delivered (e.g., R-lipoic acid or R/S lipoic acid), the weight, physical condition and age of the subject, the route of administration, and the desired final concentration of lipoic acid in the blood of the subject. Thus, the amount of lipoic acid included in a given unit dosage form can vary widely, and depends upon factors such as the species, age, weight and physical condition of the subject, as well as the method of administration. Such factors can be determined by one of skill in the art.

The data obtained from subject studies can be used in formulating a range of dosage for use in humans. Animal models, in particular sheep and pig models, are available for experimentally evaluating the compositions of the present invention. These models are commonly accepted models for the study of human fertility. Such models may also be used to evaluate whether a derivative of lipoic acid has the same function as lipoic acid as described herein. When formulating a range of dosage for use in farm animals, the farm animal may be used in trials. Methods for evaluating the impact of therapies on reproductive variables are routine.

The present invention provides methods of using the compositions described herein. Without intending to be limited by theory, it is believed that administration of lipoic acid causes a decreased clearance of progesterone from the blood of a subject, thereby allowing the subject to maintain higher levels of progesterone. In one embodiment, the present invention provides methods for decreasing progesterone clearance from the blood of a subject. In one embodiment, the present invention provides methods for increasing the concentration of progesterone in the blood of a subject. In one embodiment, the present invention provides methods for increasing the reproductive capability of a subject. Improved reproductive capability includes, but is not limited to, increased fertility and increased fecundity. As used herein, “fertility” refers to the ability to produce offspring. Increased fertility may include increased survival of embryos, such as during early embryogenesis. As used herein, “fecundity” refers to the efficiency of an individual in production of young. Animals that bring forth young frequently, regularly, and, in case of those that bear more than one offspring at a birth, in large numbers, are said to be fecund. In one embodiment, the present invention provides methods for promoting growth of an oocyte, zygote, blastocyst, embryo, and/or fetus in a subject. In one embodiment, the present invention includes methods for reducing the risk of ectopic pregnancy. In one embodiment, the present invention includes methods for reducing the risk of miscarriage.

The subject may be a vertebrate, such as a mammal. The subject can be, for instance, porcine (including, for instance, swine), bovine (including, for instance, cattle or bison), caprine (including, for instance, goats), ovine (including, for instance, sheep), equine (including, for instance, horses), feline (for instance, domestic cats, and wild cats such as tiger, lion, jaguar, leopard, cougar, cheetah, and lynx), canine, (including, for instance, domestic dogs, wolves, and foxes), members of the family Ursidae (including, for instance, pandas), members of the family Cervidae (including, for instance, deer, elk, moose, caribou and reindeer), murine (including, for instance, mice and rats), or primates (including, for instance, humans and gorillas).

A method of the present invention includes administering to a female subject in need thereof an effective amount of lipoic acid. The lipoic acid may be present in a composition. A daily dosage of lipoic acid may be at least 10 mg/kg body weight (mg/kg BW), at least 20 mg/kg BW, at least 30 mg/kg BW, at least 40 mg/kg BW, at least 50 mg/kg BW, at least 60 mg/kg BW, at least 70 mg/kg BW, at least 80 mg/kg BW, at least 90 mg/kg BW, or at least 100 mg/kg BW. Subjects (including humans) in need of lipoic acid include, but are not limited to, fertile and certain infertile subjects, those undergoing in vitro fertilization (IVF) and those not undergoing IVF, those entering a breeding program, those having an increased risk of miscarriage (e.g., those who have suffered a previous miscarriage), and older humans (e.g., those over 35 years of age). The administration may be daily (e.g., over a 24 hour period), over a 36 hour period, or over a 48 hour period.

In one embodiment, the methods include administration that begins before impregnation. In one embodiment administration may begin during the ovulatory phase, for instance, when a mature egg is released from the ovary. In one embodiment, if impregnation is to occur by IVF, administration may begin before IVF, when IVF begins, or after IVF. In one embodiment, for instance, in the case of farm animals, administration may begin when the animal enters a breeding program, such as the beginning of estrus synchronization or at the beginning of breeding. Administration may continue for at least 1 week after impregnation, at least 2 weeks after impregnation, at least 3 weeks after impregnation, at least 4 weeks after impregnation, at least 5 weeks after impregnation, at least 10 weeks after impregnation, at least 15 weeks after impregnation, at least 20 weeks after impregnation, at least 25 weeks after impregnation, at least 30 weeks after impregnation, or at least 35 weeks after impregnation. In one embodiment, administration may continue up to parturition. In one embodiment, for instance with cattle, administration may begin during a period from day −14 (two weeks before the start of estrus synchronization), from day −7 (one week before the start of estrus synchronization), or when estrus synchronization is started, and may continue to at least 1 week after impregnation, at least 2 weeks after impregnation, at least 3 weeks after impregnation, at least 4 weeks after impregnation, or at least 5 weeks after impregnation. In one embodiment, administration may begin after impregnation, such as, for example, at least 2 weeks, at least 3 weeks, at least 4 weeks after impregnation, up to parturition. Administration well after impregnation may be appropriate if a pregnant female's progesterone level drops below an acceptable range.

As used herein, the term “effective amount” refers to an amount of lipoic acid that may be effective to achieve a particular biological result. A biological result may be, for instance, decreasing progesterone clearance from the blood of a subject, increasing the concentration of progesterone in the blood of a subject, increasing the reproductive capability of a subject, promoting growth of an oocyte, zygote, blastocyst, embryo, and/or fetus in a subject, reducing the risk of ectopic pregnancy in a subject, and/or reducing the risk of miscarriage. In one embodiment, a biological result is maintaining a level of progesterone in the blood of a subject. Pregnancy can result in decreased levels of progesterone, and it can be advantageous to maintain progesterone levels at acceptable levels during pregnancy. Acceptable levels of progesterone vary depending on the species and the week of pregnancy, and the levels are known to the person of ordinary skill. In one embodiment, lipoic acid is administered to maintain a level of progesterone in the blood of at least 2 nanograms/ml (ng/ml), at least 5 ng/ml, at least 10 ng/ml, at least 15 ng/ml, at least 20 ng/ml, at least 25 ng/ml, at least 30 ng/ml, at least 35 ng/ml, at least 40 ng/ml, at least 45 ng/ml, or at least 50 ng/ml. In one embodiment, lipoic acid is administered to maintain a level of progesterone in the blood of no greater than 1 milligram/ml, no greater than 800 ng/ml, no greater than 600 ng/ml, no greater than 400 ng/ml, or no greater than 200 ng/ml. In one embodiment, a method of the present invention may include determining the level of progesterone in the blood of a subject before administration of lipoic acid. If the level is judged to be low, lipoic acid may be administered. Methods for determining the level of progesterone in blood are known and routine. Determining the level of progesterone in the blood of a subject after administration of progesterone may be done between 24 hours and 48 hours after administration of lipoic acid.

The present invention also provides methods for optimizing the therapeutic efficiency of lipoic acid treatment. The method includes determining the level of progesterone in the blood of the subject. In one embodiment, the subject is pregnant. A level of progesterone that is less than a desired concentration indicates a need to increase the amount of lipoic acid subsequently administered to the subject. Optionally, a level of progesterone that is greater than a desired concentration indicates a need to decrease the amount of lipoic acid subsequently administered to the subject. In one embodiment the subject may be one that has been administered a composition that includes lipoic acid. In one embodiment, the method includes administering to the subject in need thereof a composition that includes lipoic acid. The administration may occur before the determining or after the determining. A desired level of progesterone in the blood varies depending on, for instance, the species, whether the subject is pregnant, and the week of pregnancy, and the levels are known to the person of ordinary skill. In one embodiment, the desired level of progesterone in the blood may be 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml, or 50 ng/ml. In another embodiment, the desired level of progesterone in the blood may be at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 15 ng/ml, at least 20 ng/ml, at least 25 ng/ml, least 30 ng/ml, at least 35 ng/ml, at least 40 ng/ml, at least 45 ng/ml, or at least 50 ng/ml.

As used herein, “optimizing therapeutic efficiency of lipoic acid treatment” refers to adjusting the therapeutic dosage of lipoic acid so that a concentration of progesterone is maintained. In one embodiment, the concentration of progesterone is one that is correlated with effective treatment results. The methods described herein allow the clinician and veterinarian to provide an individually optimized dosage of lipoic acid so as to achieve a target level of progesterone in a particular subject, thereby optimizing the effectiveness of lipoic acid therapy in the subject.

The present invention is illustrated by the following examples. It is to be understood that the particular examples, materials, amounts, and procedures are to be interpreted broadly in accordance with the scope and spirit of the invention as set forth herein.

### Example 1

This example describes how supplementation of lipoic acid affected progesterone clearance rate in ovariectomized ewes.

Lipoic acid is a naturally occurring compound that has been shown to modulate insulin sensitivity when used as a diet supplement. Elevated blood insulin concentrations have been shown to decrease progesterone catabolism in several species by modulating liver enzyme. We hypothesized that lipoic acid would decrease progesterone (P4) catabolism by the liver. Eight ovariectomized ewes were fed an alfalfa-grass ration at 95% of ad libitum for the duration of the experiment. Ewes were randomly assigned to lipoic acid treatment (an empty bolus administered by gavage (n=4; CON), or lipoic acid supplemented at 32 mg/kg body weight (BW). administered by gavage (n=4; LA). Progesterone was administered via controlled internal drug release (CIDR) devices (Pfizer Animal Health, New York, N.Y., USA) on day 5 to all ewes. Daily blood samples were collected from days 5 to 10. On day 10, liver biopsies were obtained from each ewe to determine CYP3A and CYP2C activity. On day 11, serial blood samples were collected after CIDR removal to determine P4 clearance from the blood stream. Ewes treated with LA had decreased (P<0.03) serum P4 clearance compared to CON ewes; however no difference (P>0.20) in hepatic enzyme activity was found. We conclude that while lipoic acid decreases P4 clearance in the blood, it does so without affecting hepatic enzyme activity. The mechanism of action is yet to be elucidated.

### Materials and Methods

Eight Katandin cross ewes were fed an alfalfa grass mix at 95% ad libitum intake.

Day 1: treatment groups were randomly assigned and administered daily by gavage.

Control (CON; n=4): empty bolus;

Lipoic acid (LA; n=4): 32 mg/kg body weight lipoic acid. A racemic mixture of lipoic acid 50/50 of R-LA and S-LA was purchased from MTC Industries, Inc., (Edgewood, N.Y.).

Day 5: CIDR placed and daily blood samples obtained on day 5-9.

Day 10: liver biopsies performed to determine CYP3C and CYP2A enzyme activity.

Day 11: CIDRs removed and serial blood samples obtained to measure P4 catabolism.

The fractional rate constant of P4 clearance was determined using A=(A)₀e^{-kt}, where A is P4, k is the first-order fractional rate constant, and t is time.

Data were analyzed using GLM procedures of SAS and presented as LSM.

### Results

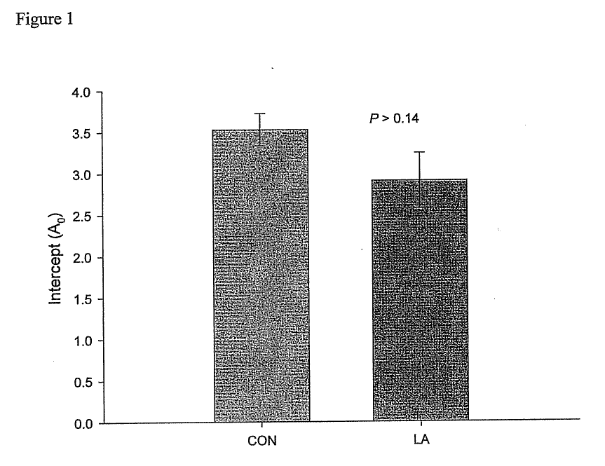

As shown in FIG. 1, there were no differences (P>0.14) found in the intercept between lipoic acid and control ewes, indicating the two groups achieved the same level of progesterone prior to removal of the CIDR.

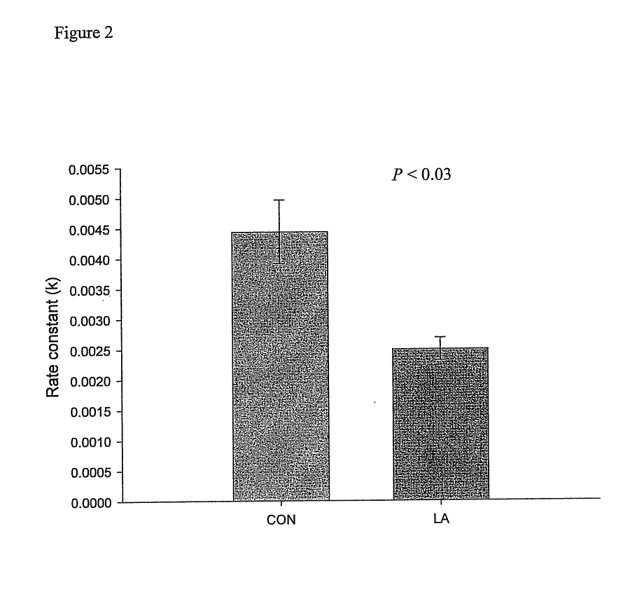

FIG. 2. Average rate constant for P4 clearance in CON and LA supplemented ovariectomized ewes after CIDR removal on d 11. As shown in FIG. 2, ewes receiving lipoic acid had a lower rate constant (P<0.03) than control ewes.

TABLE 1
Hepatic enzyme CYP2C and CYP3A activity
(pmol/min*mU) on day 10.

| | CON | LA | SEM | P-value |
|---|---|---|---|---|
| CYP2C | 25.59 | 24.13 | 4.82 | 0.84 |
| CYP3A | 10.12 | 6.27 | 2.78 | 0.37 |

No differences (P > 0.20) were found in hepatic enzyme activity.

### Conclusions

Lipoic acid decreases progesterone clearance in the blood, and it does so without affecting hepatic enzyme activity.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising administering to a subject in need thereof a composition comprising lipoic acid, wherein at least 10 milligrams lipoic acid per kilogram of body weight is administered.

2. The method of claim 1 wherein the subject is a female vertebrate.

3. The method of claim 2 wherein the vertebrate is a human or cow.

4. The method of claim 1 wherein the composition is administered orally.

5. The method of claim 1 wherein the lipoic acid is administered in a single dosage.

6. The method of claim 1 wherein the lipoic acid is administered over a 24 hour period.

7. The method of claim 1 wherein the subject is pregnant.

8. The method of claim 1 wherein the subject is part of a breeding program.

9. A method comprising administering to a female subject in need thereof an effective amount of a composition comprising lipoic acid, wherein concentration of progesterone in the blood of the subject is increased compared to the subject prior to administration of the composition.

10. The method of claim 9 wherein the concentration of progesterone in the blood is at least 2 ng/ml.

11. The method of claim 9 wherein the subject is a human or cow.

12. The method of claim 9 wherein the subject is pregnant.

13. The method of claim 9 wherein the subject is part of a breeding program.

14. The method of claim 9 wherein the reproductive capacity of the subject is increased.

15. A method for optimizing the therapeutic efficiency of lipoic acid treatment comprising:
determining the level of progesterone in the subject administered a composition comprising lipoic acid,
wherein the level of progesterone of less than a desired concentration indicates a need to increase the amount of lipoic acid subsequently administered to the subject, and
wherein the level of progesterone greater than a desired concentration indicates a need to decrease the amount of lipoic acid subsequently administered to the subject.

16. The method of claim 15 further comprising administering to the subject a composition comprising lipoic acid.

17. The method of claim 15 further comprises obtaining a biological sample from the subject.

18. The method of claim 17 wherein the biological sample comprises blood.

19. The method of claim 15 wherein the desired concentration is 2 ng/ml.

20. The method of claim 15 wherein the subject is pregnant.
